(19) United States
(12) Patent Application Publication
(10) Pub. No.: US 2009/0264494 A1
(43) Pub. Date: Oct. 22, 2009

(54) USE OF NEUROPROTECTIVE 3-SUBSTITUTED INDOLONE COMPOSITIONS

(75) Inventors: Santosh R. D'Mello, Dallas, TX (US); Edward Biehl, Dallas, TX (US)

Correspondence Address: CHALKER FLORES, LLP, 2711 LBJ FRWY, Suite 1036, DALLAS, TX, 75234, US

(73) Assignees: BOARD OF REGENTS, THE UNIVERSITY OF TEXAS SYSTEM, Austin, TX (US); SOUTHERN METHODIST UNIVERSITY, Dallas, TX (US)

(21) Appl. No.: 12/465,495

(22) Filed: May 13, 2009

Related U.S. Application Data
(63) Continuation-in-part of application No. 10/688,759, filed on Oct. 17, 2003.
(60) Provisional application No. 60/419,439, filed on Oct. 18, 2002.
(60) Provisional application No. 60/440,177, filed on Jan. 15, 2003.

(51) Int. Cl. A61K 31/404; C12N 9/99; C07D 209/34

(52) U.S. Cl. 514/414; 514/418; 435/184; 204/157.71

(57) ABSTRACT

The present invention include providing a therapeutically effective amount of a 3-substituted indolin-2-one compositions to protect against neurodegeneration including diseases such as Alzheimer's disease, Parkinson's disease, or Huntington's disease, and conditions such as ischemic stroke

### CROSS-REFERENCE TO RELATED APPLICATIONS

This application is a continuation-in-part and claims priority based on U.S. patent application Ser. No. 10/688,759, filed Oct. 17, 2003, which claims priority to U.S. Provisional Application Ser. No. 60/419,439, filed Oct. 18, 2002 and which claims priority to U.S. Provisional Application Ser. No. 60/440,177, filed Jan. 15, 2003, the entire contents of which are incorporated herein by reference.

### STATEMENT OF FEDERALLY FUNDED RESEARCH

This invention was made with U.S. Government support under Contract No. 1R01 NS047201 awarded by the National Institutes of Health and HR0011-06-1-0032 awarded by the Defense Advanced Research Projects Agency. The government has certain rights in this invention.

### TECHNICAL FIELD OF THE INVENTION

The present invention relates in general to the field of neuroprotective compositions, their synthesis and uses, and in particular to 3-substituted indolin-2-ones and their neuroprotective activity.

### INCORPORATION-BY-REFERENCE OF MATERIALS FILED ON COMPACT DISC

None.

### BACKGROUND OF THE INVENTION

Without limiting the scope of the invention, its background is described in connection with neuroprotective compositions, and in particular to 3-substituted indolin-2-ones.

Neurodegenerative pathologies including diseases such as Alzheimer's disease, Parkinson's disease, or Huntington's disease, and conditions such as ischemic stroke affect 10 millions of individuals annually and exert an enormous financial burden on society. A hallmark of these conditions is the abnormal and excessive loss of neurons. There are currently no effective strategies to prevent the neuronal death in these pathologies. In addition, neurodegenerative diseases are a major health problem particularly among the elderly. Drugs to prevent or slow down the death of neurons are urgently needed but are currently unavailable.

### SUMMARY OF THE INVENTION

The present inventors synthesized and screened different 3-substituted indolones for the ability to prevent neuronal death using a tissue culture paradigm of neurodegeneration. These investigations have led to the identification of several compounds that protected neurons from dying. These 3-substituted indolones and derivatives of them can prevent degeneration of susceptible neuronal populations in the brain, and would hence represent a therapeutic approach to treat neurodegenerative conditions. There is currently no effective strategy to cure or treat neurodegenerative diseases. These compounds represent a powerful and novel therapeutic tool.

One embodiment of the present invention provides methods and compositions used to treat neurodegenerative conditions. For example, neurodegenerative conditions may include diseases such as Alzheimer's disease, Parkinson's disease, ALS, and Huntington's disease. In addition, other neurological conditions involving neuronal loss such as ischemic stroke, traumatic brain injury and include developmental neurological disorders involving aberrant neuronal loss are also contemplated herein.

Another embodiment of the present invention provides methods and compositions used to treat diseases involving deregulation of apoptosis including cancer, autoimmunodiseases, AIDS and other diseases of the immune system. Similarly, the present invention provides methods and compositions used to treat conditions that are affected by kinases, e.g., inhibition of kinases whose activation is detrimental to neuronal survival. The present invention provides methods and compositions used to affect kinase activation of c-Raf, B-Raf, LRRK2, GSK3alpha, GSK3beta, CDK1, CDK2, and CDK4.

The present invention provides a method for inhibiting the neurodegeneration process by providing a therapeutically effective amount of the composition

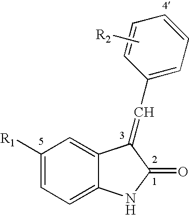

wherein R¹ is selected from the group consisting of H, halogen, nitro, and alkoxy; and R² is selected from the group consisting of 3′,5′-Br-4′-OH, 3′,5′-Br, at least one alkoxy, 2′,6′-Cl, 2-NO₂, acetyl ester, H, 4′-CH₃, 4′-OMe 4′-NMe₂, CH═CH—C₆H₅, CH═CH—C6-H4-2′NO₂, CH═CH—C₆H₄, 4′-Me, and 4′-NMe₂.

The present invention provides a method for inhibiting the neurodegeneration process by providing a therapeutically effective amount of the composition

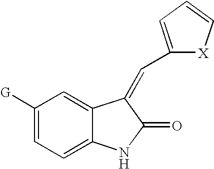

wherein G is H, Cl, Br, COMe, NO₂, F and X is NH, S or O.

The present invention provides a pharmaceutical composition for inhibiting the neurodegeneration process. The pharmaceutical composition includes a therapeutically effective amount a composition having the formula:

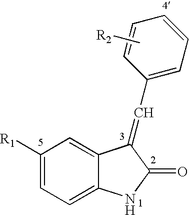

wherein R¹ is selected from the group consisting of H, halogen, nitro, and alkoxy, and R² is selected from the group consisting of 3′,5′-Br-4′-OH, 3′,5′-Br, at least one alkoxy, 2′,6′-Cl, 2-NO₂, acetyl ester, H, 4′-CH₃, 3′, 4′,5′-OMe, 4′-OMe 4′-NMe₂, CH═CH—C₆H₅, CH═CH—C6-H4-2′NO₂, CH═CH—C₆H₄, 4′-Me, and 4′-NMe₂ disposed in a pharmaceutical carrier.

The present invention provides a pharmaceutical composition for inhibiting the neurodegeneration process that includes a therapeutically effective amount a composition having the formula:

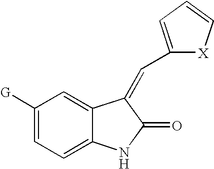

wherein G is H, Cl, Br, COMe, NO₂, F and X is NH, S or O, disposed in a pharmaceutical carrier.

The present invention provides a method for inhibiting a serine/threonine-specific kinase by providing a therapeutically effective amount of a composition selected from (E)-3-(Furan-2′-ylmethylene)indoline-2-one, (Z)-5-Bromo-3-(thien-2′-ylmethylene)indolin-2-one, (Z)-5-Nitro-3-(thien-2′-ylmethylene) indolin-2-one, (Z)-3-(Thien-2′-ylmethylene)indolin-2-one, Z)-5-Nitro-3-(1H-pyrrol-2-yl)methylene)indolin-2-one, (Z)-5-Bromo (3-(1H-pyrrol-2′-yl)methylene)indolin-2-one, (Z)-5-Chloro-(1H-pyrrol-2′-yl)methylene)indoline-2-one, (Z)-3-(1H-Pyrrol-2′-yl)methylene)indolin-2-one, (Z)-5-Acetyl-3-(3′,5′-dibromo-4′-hydroxybenzylidene)indolin-2-one, (E)-3-Benzylidene-5-fluoroindolin-2-one, (Z)-5-Fluoro-3-(3′, 4′, 5′-trimethoxybenzylidene)indolin-2-one, (Z)-3-(3′,5′-Dibromo-4-hydroxybenzylidene)-5-nitroindolin-2-one, (E)-5-Bromo-3-(4′-(dimethylamino)benzylidene) indolin-2-one, (E)-5-Bromo-3-(4′-methoxybenzylidene)indolin-2-one, (Z)-5-Bromo-3-(3′,5′-dibromobenzylidene)indolin-2-one, (E)-5-Bromo-3-(3′,5′-dibromo-4-hydroxybenzylidene)indolin-2-one, (E)-5-Chloro-3-(4′-(dimethylamino)benzylidene)indolin-2-one, (E)-5-Chloro-3-(4′-methoxybenzylidene)indolin-2-one, (E)-5-Chloro-3-(4′-methybenzylidene)indolin-2-one, (E)-3-Benzylidene-5-chloroindolin-2-one, (E)-5-Chloro-3-(2′,6′-dichlorobenzylidene)indolin-2-one, (Z)-5-Chloro-3-(3′, 4′, 5′-trimethoxybenzylidene)indolin-2-one, (Z)-3-(3′,5′-Dibromobenzylidene)-5-chloroindolin-2-one, (E)-3-(3′,5′-Dibromo-4-hydroxybenzylidene)-5-chlorondolin-2-one, (Z-3-(2′-Nitrobenzylidene)indolin-2-one, (E)-3-(2′,6′-Dichlorobezylidene)indolin-2-one, 3-(3′-Phenylallylidene)indolin-2-one, (E)-3-(3′,5′-Dibromo-4′-hydroxybenzylidene)indolin-2-one, GW5074, IC261, DMBI, GW8510, VEGFR-2 Inh I, VEGFR-2 Inh II, and SU6656.

The present invention provides a method for the microwave synthesis of indolinones by mixing an optionally substituted aldehyde with an optionally substituted indolin-2-one and piperidene; exposing the mixture to microwaves and collecting a precipitated composition.

One embodiment of the present invention includes a composition having the structure listed below:

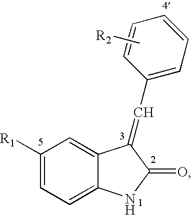

wherein R¹ is selected from H, Cl, Br, NO₂, F, COMe, and R² is selected from 3′,5′-Br-4′-OH, 3′,5′-Br, 3′,4′,5′-OMe, 2′,6′-Cl, 2-NO₂, 3′,5′-Br-4′-OAc, H, 4′-CH₃, 4′-OMe 4′-NMe₂, 3′,4′,5′-OMe, CH═CH—C6-H4-2′NO₂, CH═CH—C₆H₄, 4′-Me, and 4′-NMe₂,

Another embodiment of the present invention includes a 3-(hetarylmethylene) indolin-2-ones composition having the structure listed below:

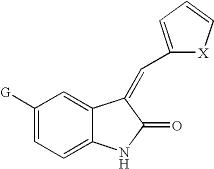

wherein G is H, Cl, Br, COMe, NO₂, F and X is NH, S or O.

### BRIEF DESCRIPTION OF THE DRAWINGS

For a more complete understanding of the features and advantages of the present invention, reference is now made to the detailed description of the invention along with the accompanying figures in which:

FIG. 1 is a photograph illustrating the qualitative neuroprotective effects of compounds using phase-contrast micrographs of neuronal morphology, DAPI staining of nuclei showing fragmented nuclei, TUNEL staining also showing fragmented DNA, and active caspase-3 immunoreactivity highly indicative of apoptosis.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, an indole may be used interchangeably with the term 2,3-Benzopyrrole, ketole, and 1-benzazole to denote an aromatic heterocyclic organic compound. It has a bicyclic structure, consisting of a six-membered benzene ring fused to a five-membered nitrogen-containing pyrrole ring with a nitrogen lone electron pair in the aromatic ring.

As used herein, the term “pharmaceutical composition” also means a solution, suspension, cream, ointment, lotion, capsule, caplet, softgel, gelcap, suppository, enema, elixir, syrup, emulsion, film, granule, gum, insert, jelly, foam, paste, pastille, pellet, spray, troche, lozenge, disk, magma, poultice, or wafer and the like. In addition the pharmaceutical composition may include other additives conventionally used in pharmaceutical compositions and known to those of skill in the art, e.g., anti-adherents, anti-sticking agents, glidants, flow promoters, lubricants, talc, magnesium stearate, fumed silica), micronized silica, surfactants, waxes, stearic acid, stearic acid salts, stearic acid derivatives, starch, hydrogenated vegetable oils, sodium benzoate, sodium acetate, leucine and magnesium lauryl sulfate. Other additives may be incorporated into the immediate-release layer, such as diluents, binders, lubricants, antioxidants, colourings, sweeteners, flavourings and acidulants, wetting agents, hydrophilizing agents such as sorbitol and cyclodextrins, osmotic agents such as mannitol, pH correctors, stabilizing agents such as trehalose and mannitol, adsorbants, chelating and sequestering agents and gastroresistant film-coating excipients of the type including cellulose acetylphthalate and polymethacrylates.

As used herein, the term “therapeutically effective amount” is meant an amount of a compound of the present invention effective to yield a desired therapeutic response. For example to prevent or treat the symptoms in a host or an amount effective to treat the condition. The specific “therapeutically effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “pharmaceutical carrier” is a pharmaceutically acceptable solvent, suspending agent or vehicle for delivering the anti-cancer agent to the animal or human. The carrier may be liquid or solid and is selected with the planned manner of administration in mind.

As used herein, “a subject in need thereof” is a patient, animal, mammal or human, who will benefit from the method of this invention. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

As used herein, the term “optional” and “optionally” denotes that the subsequently described event or circumstance may or may not occur. As used herein, the term “alkyl” denotes branched or unbranched hydrocarbon chains, preferably having about 1 to about 10 carbons. As used herein, the term “Alkenyl” denotes optionally substituted straight chain and branched hydrocarbon radicals having about 1 to about 10 carbons with at least one carbon-carbon double bond. As used herein, the term “aryl” denotes a chain of carbon atoms which form a ring system with at least one aromatic ring having between about 4-14 carbon atoms. As used herein, the term “Alkoxy” denotes an alkyl ether group and includes an optionally substituted straight chain or branched alkyl group having about 1 to about 20 carbons with a terminal oxygen linking the alkyl group to the rest of the molecule. As used herein, the term “Heterocyclyl” denotes optionally substituted aromatic and nonaromatic rings having carbon atoms and at least one heteroatom (O, S, N) or heteroatom moiety (SO₂, CO, CONH, COO) in the ring. As used herein, the term “Acyl” denotes to a carbonyl moiety attached to either a hydrogen atom (i.e., a formyl group) or to an optionally substituted alkyl or alkenyl chain, or heterocyclyl. As used herein, the term “Aroyl” denotes to a carbonyl moiety attached to an optionally substituted aryl or heteroaryl group, wherein aryl and heteroaryl have the definitions provided above. In particular, benzoyl is phenylcarbonyl.

Another embodiment of the present invention provides methods and compositions used to treat diseases involving deregulation of apoptosis including cancer, autoimmunodiseases, AIDS and other diseases of the immune system. Similarly, the present invention provides methods and compositions used to treat conditions that are affected by kinases, e.g., inhibition of kinases whose activation is detrimental to neuronal survival.

The present invention provides methods and compositions used to affect kinase activation of c-Raf, B-Raf, LRRK2, GSK3alpha, GSK3beta, CDK1, CDK2, and CDK4. Raf is family of genes that code for a protein kinases, e.g., c-Raf and B-Raf. The c-Raf protein functions in the MAPK/ERK signal transduction pathway as part of a protein kinase cascade as a serine/threonine-specific kinase. c-Raf is a MAP kinase (MAP3K) which functions downstream of the Ras family of membrane-associated GTPases to which it binds directly. Once activated c-Raf can phosphorylate to activate the dual specificity protein kinases MEK1 and MEK2 which in turn phosphorylate to activate the serine/threonine specific protein kinases ERK1 and ERK2. Activated ERKs are pleiotropic effectors of cell physiology and play an important role in the control of gene expression involved in the cell division cycle, apoptosis, cell differentiation and cell migration.

The compound number identifies the Compound referenced in the FIGURES and the tables herein. Unless otherwise noted, all products were obtained as crude solids which were purified by recrystallization from ethanol. NMR spectra were recorded by Brucker-400 MHz and JEOL-500 MHz Spectrometer. Chemical shifts are reported in parts per million (d) downfield from TMS. Coupling constants are reported in hertz (Hz). Elemental analyses were performed on a Thermo Finnigan CE Elantech Model Flash EA1 112 elemental analyzer with a Model MAS200R auto sampler. Observed C, H, and N elemental analysis of all compounds were within ±0.4% of calculated values. Melting points were taken on a Mel-Temp apparatus and are uncorrected. NMR spectra were recorded on a Brucker-400 MHz and/or JEOL-500 MHz Spectrometer. Chemical shifts are reported in parts per million downfield from TMS. Coupling constants are reported in hertz (Hz). Chemicals were purchased from Sigma Aldrich chemical company and were used as received. HRMS analyses were performed by the Washington University Center for Biomedical and Bioorganic Mass Spectrometry.

Chemical Synthesis of 3-(Benzylidene)indolin-2-ones. As shown in Table 1, a variety of 3-benzylidenes (1-6) and 5-substituted (3-benzylidene)indolin-2-ones (Compounds 7-34). The 3-benzylidene derivatives (Compounds 1, 7, 16, 21, 30, and 34) were prepared according to method of Andreani et al. (Method A). For example, 3′,5′-dibromo-4′-hydroxybenzaldehyde (2 mmol) was treated with the appropriate indolinone (2 mmol), anhydrous sodium acetate (4 mmol) in 20 ml of acetic acid. After the reaction mixture was refluxed for 3 hours, it was cooled and evaporated under reduced pressure. The residue was poured onto crushed ice, and the resulting precipitate was collected by filtration (70-80% yields) and recrystallized from ethanol.

TABLE 1

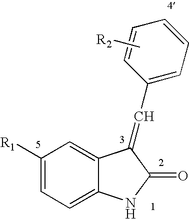

| | | | NMR data | | | | | |
|---|---|---|---|---|---|---|---|---|
| | | | Chemical shift, ppm | | | % neuronal survival | | |
| Substituents | | | %, Z | Z isomer | E isomer | Mean ± SD | | |
| ID | R₁ | R₂ | isomer | 2′,6′-H | 2′,6′-H | 1 μM | 5 μM | 25 μM |
| 1 | H | 3′,5′-Br-4′-OH | 10^{a} | 8.78 | 7.87 | 69.7 ± 2.19* | 88.4 ± 11.74* | 84.6 ± 7.57* |
| 2 | H | 3′,5′-Br | 10 | 8.69 | 7.85 | 54.7 ± 5.32 | 45.4 ± 4.32 | 46.9 ± 12.30 |
| 3 | H | 3′,4′,5′-OMe | 90 | 7.98 | 7.03 | 50.8 ± 7.50 | 59.9 ± 7.98 | 61.3 ± 11.35 |
| 4 | H | CH—CH—C₆H₄ | 50 | 7.75 | 7.40 | 86.0 ± 2.03* | 83.6 ± 18.88 | 81.1 ± 6.22* |
| 5 | H | 2′,6′-Cl | 0 | — | — | 54.6 ± 10.27 | 52.9 ± 3.94 | 24.5 ± 12.38 |
| 6 | H | 2-NO₂ | 80 | 7.76 | 6.98 | 75.8 ± 3.20* | 76.6 ± 7.14 | 81.5 ± 13.72 |
| 7 | Cl | 3′,5′-Br-4′-OH | 5^{b} | 8.74 | 7.87 | 84.3 ± 1.04* | 91.9 ± 5.31* | 81.8 ± 14.94* |
| 8 | Cl | 3′,5′-Br | 90 | 8.59 | 7.79 | 77.7 ± 17.51 | 68.5 ± 11.86 | 64.4 ± 9.65 |
| 9 | Cl | 3′,5′-Br-4′-OAc | 20 | 8.49 | 7.84 | 73.0 ± 5.59* | 84.6 ± 16.19* | 75.2 ± 31.54 |
| 10 | Cl | 3′,4′,5′-OMe | 80 | 7.97 | 7.02 | 81.1 ± 9.81* | 71.0 ± 7.26* | 57.1 ± 10.13 |
| 11 | Cl | 2′,6′-Cl | 0 | — | — | 56.4 ± 14.41 | 63.8 ± 3.46 | 45.7 ± 24.05 |
| 12 | Cl | H | 6 | 8.30 | 7.69 | 58.6 ± 3.27 | 63.8 ± 8.63 | 41.1 ± 21.41 |
| 13 | Cl | 4′-CH₃ | 20 | 8.49 | 7.56 | 70.0 ± 17.50 | 70.6 ± 13.86 | 67.4 ± 17.40 |
| 14 | Cl | 4′-OMe | 4 | 8.55 | 7.69 | 84.9 ± 6.28* | 85.6 ± 4.49* | 81.6 ± 9.09* |
| 15 | Cl | 4′-NMe₂ | 4 | — | 7.63 | 51.8 ± 15.66 | 55.8 ± 16.94 | 79.7 ± 4.69* |
| 16 | Br | 3′,5′-Br-4′-OH | 5^{c} | 8.74 | 7.55 | 88.4 ± 0.71* | 98.7 ± 3.85* | ^{e} |
| 17 | Br | 3′,5′-Br | 90 | 8.59 | 7.57 | 54.7 ± 8.56 | 45.4 ± 3.05 | 46.9 ± 7.74 |
| 18 | Br | 3′,4′,5′-OMe | 90 | 8.01 | 7.06 | 50.8 ± 7.67 | 59.9 ± 12.94 | 61.3 ± 1.20 |
| 19 | Br | 4′-OMe | 10 | 8.45 | 7.66 | 57.1 ± 4.51 | 59.5 ± 9.10 | 60.5 ± 9.56 |
| 20 | Br | 4′-NMe₂ | 10 | 8.43 | 7.59 | 56.5 ± 2.28 | 54.9 ± 9.18 | 68.6 ± 9.38 |
| 21 | N | 3′,5′-Br-4′-OH | 70^{d} | 8.77 | 8.01 | 79.9 ± 12.81 | 78.4 ± 31.66 | 71.3 ± 19.80 |
| 22 | N | 3′,5′-Br | 90 | 8.63 | 7.72 | 78.1 ± 5.82* | 78.7 ± 15.86 | 74.5 ± 9.12 |
| 23 | N | 3′,5′,4′-OMe | 90 | 8.07 | 7.16 | 76.6 ± 15.98 | 61.9 ± 5.87 | 69.0 ± 11.26 |
| 24 | N | 2′,6′-Cl | 0 | — | — | 76.4 ± 9.84* | 69.9 ± 11.25 | 70.7 ± 8.50 |
| 25 | N | H | 90 | 8.40 | 7.33 | 80.1 ± 18.01 | 78.4 ± 17.54 | 54.3 ± 3.63 |
| 26 | N | CH═CH—C₆H₅ | — | 7.79 | 7.59 | 80.1 ± 4.21* | 78.6 ± 13.04 | 83.9 ± 9.28* |
| 27 | N | CH═CH—C₆— | 0 | — | — | 78.6 ± 6.84* | 51.9 ± 7.50 | 78.6 ± 16.33 |
| 28 | N | 4′-Me | 90 | 8.33 | 7.67 | 54.6 ± 7.48 | 60.0 ± 1.87 | 54.3 ± 0.49 |
| 29 | N | 4′-NMe₂ | 90 | 8.48 | 7.68 | 70.4 ± 9.37 | 67.6 ± 10.31 | 66.9 ± 9.86 |
| 30 | F | 3′,5′-Br-4′-OH | 5^{c} | 8.74 | 7.87 | 49.3 ± 10.34 | 69.6 ± 8.96 | 18.1 ± 11.00* |
| 31 | F | 3′,5′-Br | 30 | 8.59 | 7.88 | 38.4 ± 6.24 | 46.1 ± 4.00 | 48.1 ± 14.65 |
| 32 | F | 3′,4′,5′-OMe | 70 | 7.98 | 7.02 | 59.2 ± 1.77 | 51.9 ± 7.14 | 31.9 ± 1.77 |
| 33 | F | H | 10 | 8.35 | 7.65 | 53.4 ± 2.12 | 51.9 ± 15.56 | 31.5 ± 17.51 |
| 34 | COMe | 3′,5′-Br-4′-OH | 90^{d} | 8.80 | 7.96 | 96.8 ± 6.15* | 110.3 ± 8.50* | 2.2 ± 1.63* |

^{a}Changed to 50% Z after 6 hours.
^{b}Changed to 70% Z after 6 hours.
^{c}Changed to 80% Z after 6 hours.
^{d}No change after 6 hours.
^{e}Could not be accurately evaluted because of precipitation of the compound in the culture medium.
*P < 0.05 compared with viability of culture receiving LK

The remaining Compounds 2-6, 8-15, 17-20, 22-29, and 31-33, were prepared by the method of Sun et al. (Method B). In a typical experiment, the appropriate aldehyde (1 mmol) was dissolved in ethanol (10 mL) and treated with the equivalent of the corresponding indolinone (1 mmol) and piperidine (0.1 mmol). The reaction mixture was refluxed for 3-5 hours then cooled to room temperature. During that time a precipitate formed which was collected by filtration (80-95% yield) and recrystallized from ethanol.

Also listed in Table 1 are the % of Z isomer and chemical shift of the 2′,6′-H of the Z and E isomers. The ¹H NMR spectra are in agreement with assigned structures as determined by NOE for all compounds having a 2′-H or 2′,6′-H. As shown in Table 1, the chemical shifts of the 2′,6′-H in the Z configuration ranged from 7.76 to 8.78 and from 7.59-8.80 for the E configuration. In the case of the 2′,6′-dichloro derivative (Compound 11), X-ray analysis confirmed that Compound 11 existed in the E configuration. Interestingly, with the exception of the 5-nitro, 5-aceto and 3-(2′,6′)-dichloro derivatives, the other benzylidenes were unstable in DMSO-d6 changing slowly to give mixtures of Z and E isomers. The 3′,5′-dibromo-4′-hydroxy derivatives (Compounds 1, 7, 16, 21, 30 and 34) are readily soluble in DMSO-d6 and with the exception of the 5-nitro (Compound 21) and the 5-aceto (Compound 34) derivatives, the initial (within 5 minutes of preparation) NMR spectra were mainly in the E configuration but slowly changed to give mixtures of E and Z isomers mainly in favor of the Z isomer. Thus, the percent of Z configuration of Compound 1 changed from 10% to 50% after 6 hours from mixing, whereas the percent of Z configuration of Compounds 7 and 16 changed from 5% to 70% and from 5% to 80%, respectively. In the exceptional cases, Compound 21 existed as a 70:30 mixture of Z and E isomers, respectively, even after standing for 6 hours.

Chemical Synthesis of 3-(Hetarylmethylene)indolin-2-ones. Table 2 provides six 3-(1 H-pyrrol-2-yl) (Compounds 34-40) and 3-(thiophene-2-yl) (Compounds 41-44) 3-(Hetarylmethylene) indolin-2-one derivatives which were prepared by Method B. The (2′-furanyl) derivative (Compound 45) was prepared by the method, and 3-(furan-2-yl) (45) which was prepared by Method C. Additionally, the proposed structures listed in Table 2 include the percentages of Z isomers and chemical shift of the vinyl hydrogen of the Z and E isomers which were obtained by NOE. As shown, the pyrrol-2-yl and 2-thienyl analogs exist exclusively in the Z configuration.

In a typical reaction a mixture containing oxindole (1 equiv), furan-2-carboxaldehyde (1.2 equiv), and piperidine (0.1 equiv) in (8 mL of methanol) was stirred at room temperature for 30 minutes. After the mixture was cooled to 0° C., the reaction was stirred overnight. The resulting precipitate was filtered, washed with cold methanol, and dried to give the target compounds (80-90% yield). The resulting compounds were recrystallized from ethanol. The melting points, spectral properties and HRMS analyses are given below for all synthesized compounds.

TABLE 2

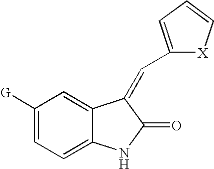

| | | | NMR data | | | % neuronal survival | | |
|---|---|---|---|---|---|---|---|---|
| Substituents | | | Chemical shift, ppm | | | Mean ± SD | | |
| ID | G | X | % Z | 1-vinyl H | H-4 | 1 μM | 5 μM | 25 μM |
| 35 | H | NH | 100 | 7.70 | 7.59 | 56.0 ± 17.21 | 51.1 ± 7.06 | 3.88 ± 9.58* |
| 36 | Cl | NH | 100 | 7.85 | 7.74 | 71.2 ± 0.02* | 84.0 ± 16.97* | 82.6 ± 10.18* |
| 37 | Br | NH | 100 | 7.86 | 7.84 | 75.5 ± 14.58 | 94.2 ± 0.15* | 93.0 ± 1.38* |
| 38 | COMe | NH | 100 | 7.95 | 8.26 | 69.5 ± 12.61 | 87.3 ± 22.50 | 87.1 ± 24.50 |
| 39 | NO₂ | NH | 100 | 8.13 | 8.56 | 84.4 ± 3.41* | 90.1 ± 6.55* | 87.9 ± 1.40* |
| 40 | F | NH | 100 | 7.85 | 7.70 | 58.4 ± 3.98 | 65.7 ± 0.07 | 96.6 ± 1.91* |
| 41 | H | S | 100 | 7.96 | 8.13 | 68.3 ± 13.16 | 77.1 ± 13.16 | 86.2 ± 4.03* |
| 42 | NO₂ | S | 100 | 8.63 | 8.52 | 75.1 ± 9.11* | 67.6 ± 2.29 | 66.9 ± 10.30 |
| 43 | Br | S | 100 | 8.22 | 7.90 | 56.8 ± 5.62 | 58.3 ± 9.82 | 66.4 ± 4.56 |
| 44 | F | S | 100 | 8.15 | 7.56 | 53.6 ± 14.24 | 60.1 ± 10.28 | 68.7 ± 9.35 |
| 45 | H | O | 0 | 7.31 | 8.35 | 70.4 ± 8.35 | 82.8 ± 5.22* | 90.2 ± 3.91* |

In addition, the indolinone compounds of the present invention may be synthesized by microwave synthesis techniques: For example, the appropriate aldehyde (1 mmol) was added to a mixture of appropriate substituted indolin-2-one (1 mmol) and piperidene (1 mmol) in ethanol (3 ml). The mixture was placed in a microwave test tube. The tube was then capped and charged into a CEM microwave instrument. The mixture was irradiated with 250 psi pressure and at a temperature of 140° C. for 5-10 minutes. Then the reaction mixture was left overnight at room temperature. The obtained solid was collected by filtration and washed with cold ethanol. The crude product was purified by recrystallisation from ethanol. The variables may be altered for the specific reaction conditions. For example, the pressure may be 100-500 psi and the temperature of may vary from 100-200° C. and the duration may be from 1 minute to several hours.

As used herein, Compound 1 is (E)-3-(3′,5′-Dibromo-4′-hydroxybenzylidene)indolin-2-one which may be prepared by Method A; solid; in yield 76%; mp 238-241° C. ¹H NMR (500 MHz, DMSO-d₆)™ 10.56 (s, 1H, NH-1), 7.87 (s, 2H, H-2,6′),), 7.45 (d, J=4.60 Hz, 2H, H-vinyl, H-7) 7.45 (d, J=4.60 Hz, 2H, H-4,7), 7.20 (t, J=7.45 Hz, 1H. H-6), 6.85 (t, J=6.25 Hz, 1H, H-5); NOE effect between H-vinyl and H-2,6′. ¹³C NMR (500 MHz, DMSO-d₆)™168.9 (CO), 152.3 (C), 143.5 (C), 133.7 (CH), 133.6 (CH) 130.8 (CH), 129.8 (C), 128.3 (C), 122.4 (CH), 121.7 (CH), 121.1 (C), 112.3 (2×C), 110.8 (CH); HRMS Calcd. for C₁₅H₉Br₂NO₂: 392.9000. Found: 392.2002.

As used herein, compound 2 is (E)-3-(3′,5′-Dibromobenzylidene)indolin-2-one which may be prepared by Method B; solid; yield 83%; mp 245-247° C. ¹H NMR (500 MHz, DMSO-d₆)™ 10.63 (s, 1H, NH-1), 7.90 (s, 1H, H-4′), 7.85 (s, 2H, H-2′, 6′), 7.49 (s, 1H, H-vinyl), 7.26 (d, J=7.45 Hz, 1H, H-4), 7.21 (t, J=7.45 Hz, 1H, H-6), 6.85 (m, 1H, H-5,7); ¹³C NMR (500 MHz, DMSO-d₆)™ 168.6 (CO), 143.9 (C), 139.2 (C), 134.2 (CH), 132.7 (CH), 131.4 (CH), 131.1 (CH), 130.3 (C), 123.2 (C), 122.8 (CH), 121.8 (CH), 120.9 (C), 110.9 (CH); HRMS Calcd. for C₁₅H₁₀Br₂NO: 377.0540. Found: 377.0534.

As used herein, compound 3 is (Z)-3-(3′, 4′, 5′-Trimethoxybenzylidene)indolin-2-one which may be prepared by Method B; solid yield 88%; mp 200-202° C. ¹H NMR (500 MHz, DMSO-d₆)™ 10.55 (s, 1H, NH-1), 7.98 (s, 2H, H-2′,6′), 7.73 (s, 1H, H-vinyl), 7.64 (d, J=7.45 Hz, 1H, H-4), 7.17 (t, J=7.45 Hz, 1H, H-6), 6.97 (t, J=7.45 Hz, 1H, H-5), 6.81 (d, J=8.05 Hz, 1H, H-7), 3.81 (s, 6H, 2×OCH₃), 3.71 (s, 3H, OCH₃); NOE effect between 4H and H-vinyl. ¹³C NMR (500 MHz, DMSO-d₆)™ 167.8 (CO), 152.7 (C), 140.9 (C), 140.2 (C), 137.8 (CH), 130.0 (C), 129.1 (CH), 126.1 (C), 125.7 (C), 121.5 (CH), 119.9 (CH), 110.6 (CH), 109.8 (CH), 60.7 (OCH₃), 56.4 (OCH₃); HRMS Calcd. for C₁₈H₁₇NO₄: 311.3319. Found: 311.3323.

As used herein, compound 4 is 3-(3′-Phenylallylidene)indolin-2-one which may be prepared by Method B; orange solid; yield 80%; mp 203-205° C. (lit. [8] 205-206° C.).

As used herein, compound 5 is (E)-3-(2′,6′-Dichlorobezylidene)indolin-2-one which may be prepared by Method B; red solid, 83% yield, mp 179-181° C., (lit. 164° C.) [8]. (Probably reported a mixture of E and Z isomers). ¹H NMR (500 MHz, DMSO-d₆)™ 10.78 (s, 1H, NH-1), 7.60 (d, J=8.60 Hz, 2H, H-3′,5′), 7.49 (t, J=8.60 Hz, 1H, H-4′), 7.38 (s, 1H, H-vinyl), 7.19 (t, J=8.60 Hz, 1H, H-5), 6.82 (d, J=8.60 Hz, 1H, H-4), 6.74 (t, J=8.0 Hz, 1H, H-6), 6.45 (d, J=8.0 Hz, 1H, H-7); ¹³C NMR (500 MHz, DMSO-d₆)™ 168.0 (CO), 143.5 (C), 133.7 (C), 132.3 (C), 131.8 (CH), 131.4 (CH), 129.1 (CH), 128.7 (CH), 123.3 (CH), 122.1 (CH), 121.2 (C), 110.7 (CH).

As used herein, compound 6 is (Z-3-(2′-Nitrobenzylidene)indolin-2-one which may be prepared by Method B; solid; yield 81%; mp 239-241° C. ¹H NMR (500 MHz, DMSO-d₆)™10.70 (s, 1H, NH-1), 8.30 (d, 1H, J=8.0 Hz, H-4), 7.76-7.92 (m, 4H, H-3′, 5′, H-vinyl), 7.29 (t, J=7.2 Hz, 1H, H-4′), 6.73-6.89 (m, 3H, H-5,6,7); NOE between H-vinyl and H-4. ¹³C NMR (500 MHz, DMSO-d₆)™ 168.5 (CO), 147.6 (C), 143.5 (C), 135.0 (CH), 133.0 (CH), 131.5 (CH), 131.1 (CH), 130.9 (CH), 129.0 (C), 125.7 (CH), 122.9 (CH), 121.7 (CH), 110.8 (CH); HRMS Calcd. for C₁₅H₁₀N₂O₃: 266.069. Found: 266.0679.

As used herein, compound 7 is (E)-3-(3′,5′-Dibromo-4-hydroxybenzylidene)-5-chlorondolin-2-one which may be prepared by Method A; solid; yield 80%; mp 190-193° C. ¹H NMR (500 MHz, DMSO-d₆)™ 10.70 (s, 1H, NH-1), 7.87 (s, 2H, H-2′,-6′), 7.49 (s, 1H, H-vinyl), 7.37 (s, 1H, H-4), 7.20 (d, J=8.0 Hz, 1H, H-7), 6.82 (d, J=8.0 Hz, 1H, H-6); NOE between H-vinyl and H-2′,6′. ¹³C NMR (500 MHz, DMSO-d₆)™ 168.6 (CO), 152.7 (C), 142.3 (C), 135.4 (CH), 133.8 (CH), 130.1 (CH), 128.7 (C), 127.2 (C), 125.4 (C), 122.9 (C), 122.2 (CH), 112.3 (C), 112.1 (CH); HRMS Calcd. for C₁₅H₈Br₂ClNO₂: 426.8610. Found: 426.8617.

As used herein, compound 8 is (Z)-3-(3′,5′-Dibromobenzylidene)-5-chloroindolin-2-one which may be prepared by Method B; solid; yield 85%; mp 294-297° C. ¹H NMR (500 MHz, DMSO-d₆)™ 10.82 (s, 1H, NH-1), 8.59 (s, 2H, H-2′, 6′), 7.90 (s, 1H, H-4′), 7.85 (s, 1H, H-vinyl), 7.75 (d, J=6.85 Hz, 1H, H-4), 7.23 (d, J=8.60 Hz, 1H, H-6), 6.81 (d, J=8.60 Hz, 1H, H-7); NOE between H-vinyl and H-4 ¹³C NMR (500 MHz, DMSO-d₆)™ 168.2 (CO), 141.9 (C), 138.2 (C), 135.4 (CH), 135.3 (CH), 133.7 (CH), 129.7 (CH), 126.5 (C), 125.5 (C), 122.7 (C), 120.9 (CH), 111.6 (CH); HRMS Calcd. for C₁₅H₉Br₂ClNO: 411.3719. Found: 411.3713.

As used herein, compound 9 is (E)-2,6-Dibromo-4-(5′-chloro-2′-oxoindolin-3-ylidene)methyl)phenyl acetate which may be prepared by Method B in 88% yield, mp 148-150° C. ¹H NMR (500 MHz, DMSO-d₆)™ 10.80 (s, 1H, NH-1), 7.84 (s, 2H, H-2′, 6′), 7.52 (s, 1H, H-vinyl), 6.68 (s, 1H, H-4), 6.64 (d, J=8.45 Hz, 1H, H-6), 6.02 (d, J=8.45 Hz, 1H, H-7); HRMS Calcd. for C₁₇H₁₀Br₂ClNO₃: 411.8719. Found: 411.8706.

As used herein, compound 10 is (Z)-5-Chloro-3-(3′, 4′, 5′-trimethoxybenzylidene)indolin-2-one which may be prepared by Method B in 90% yield, mp 269-271° C. ¹H NMR (400 MHz, DMSO-d₆)™ 10.64 (s, 1H, NH-1), 7.97 (s, 2H, H-2′,6′), 7.83 (s, 1H, H-vinyl), 7.72 (d, J=2.0 Hz, 1H, H-4), 7.15 (dd, J=8.0, 2.0 Hz, 1H, H-7), 6.76 (d, J=8.0 Hz, 1H, H-6), 3.76 (s, 6H, 2×OCH₃), 3.68 (s, 3H, OCH₃); NOE effect between H-vinyl and H-4; ¹³C NMR (400 MHz, DMSO-d₆)™ 167.9 (CO), 153.1 (C), 141.0 (C), 140.1 (CH), 139.9 (C), 130.2 (C), 128.7 (CH), 128.0 (C), 126.2 (C), 125.2 (C), 120.3 (CH), 111.5 (CH), 111.3 (CH), 61.0 (OCH₃), 56.7 (OCH₃); HRMS Calcd. for C₁₈H₁₆ClNO₄: 396.0768. Found: 396.0770

As used herein, compound 11 is (E)-5-Chloro-3-(2′,6′-dichlorobenzylidene)indolin-2-one which may be prepared by Method B in 92% yield, mp 197-199° C. ¹H NMR (400 MHz DMSO-d₆)™ 10.88 (s, 1H, NH-1), 7.69 (d, J=7.88 Hz, 2H, H-3′, 5′), 7.54-7.68 (m, 2H, H-vinyl, H-4′), 7.30 (d, J=8.0 Hz, 1H, H-4), 6.90 (d, J=8.2 Hz, 1H, H-6), 6.36 (d, J=8.2 Hz, 1H, H-7); ¹³C NMR (400 MHz, DMSO-d₆)™ 167.9 (CO), 142.6 (C), 133.9 (C), 132.7 (C), 132.5 (CH), 131.8 (C), 131.3 (CH), 131.0 (CH), 129.6 (CH), 126.2 (C), 123.1 (CH), 112.6 (CH).

As used herein, compound 12 is (E)-3-Benzylidene-5-chloroindolin-2-one which may be prepared by Method B in 94% yield, mp 208-211° C. ¹H NMR (400 MHz DMSO-d₆)™10.76 (s, 1H, NH-1), 7.68-7.73 (m, 3H, H-2′,6′, vinyl), 7.53-7.56 (m, 3H, H-3′, 5′,4), 7.49 (d, J=8.05 Hz, 1H, H-6), 7.28 (t, J=8.60 Hz, 1H, H-4′), 6.87 (d, J=8.05 Hz, 1H, H-7); NOE effect between H-vinyl and H-2,6′; ¹³C NMR (400 MHz, DMSO-d₆)™ 169.1 (CO), 142.5 (C), 138.6 (C), 134.9 (CH), 130.5 (CH), 129.7 (CH), 125.7 (CH), 123.3 (CH), 122.6 (CH), 112.4 (CH); HRMS Calcd. for C₁₅H₁₀ClNO: 255.0451. Found: 255.0459.

As used herein, compound 13 is (E)-5-Chloro-3-(4′-methybenzylidene)indolin-2-one which may be prepared by Method B in 86% yield, mp 220-223° C. ¹H NMR (500 MHz, DMSO-d₆)™ 10.69 (s, 1H, NH-1), 7.64 (s, 1H, H-vinyl), 7.56 (d, J=8.20 Hz, 2H, H-2′,6′), 7.45 (s, 1H, H-4), 7.30 (d, J=7.45 Hz, 2H, H-3′,5′), 7.23 (d, J=8.05 Hz, 1H, H-6), 6.85 (d, J=8.05 Hz, 1H, H-7), 2.35 (s, 3H, CH₃); NOE between H-vinyl and H-2′-6′. ¹³C NMR (500 MHz, DMSO-d₆)™ 168.9 (CO), 142.1 (C), 140.7 (C), 138.4 (CH), 131.6 (C), 129.9 (CH), 126.6 (C), 125.4 (C), 122.1 (C), 111.9 (CH), 21.6 (CH₃); HRMS Calcd. for C₁₆H₁₂ClNO: 269.0607. Found: 269.0611.

As used herein, compound 14 is (E)-5-Chloro-3-(4′-methoxybenzylidene)indolin-2-one which may be prepared by Method B in 87% yield, mp 257-260° C. ¹H NMR (400 MHz, DMSO-d₆)™ 10.69 (s, 1H, NH-1), 7.69 (d, J=8.48 Hz, 2H, H-2′,6′), 7.54 (d, J=8.0 Hz, 1H, H-4), 7.25 (s, 1H, H-vinyl), 7.15 (t, J=8.0 Hz, 1H, H-6), 7.10 (d, J=8.50 Hz, 2H, H-3′, 5′), 6.80-6.88 (m, 2H, H-3′, 5′), 3.83 (s, 3H, OCH₃); NOE effect between H-vinyl and H-2′,6′. ¹³C NMR (400 MHz, DMSO-d₆)™ 169.4 (CO), 161.7 (C), 142.2 (C), 139.7 (C), 138.7 (CH), 135.6 (CH), 132.4 (CH), 129.9 (CH), 127.0 (C), 125.7 (C), 125.6 (C), 123.6 (C), 122.2 (CH), 115.2 (CH), 112.2 (CH), 56.2 (OCH₃); HRMS Calcd. for C₁₆H₁₂ClNO₂: 265.0557. Found: 265.0576.

As used herein, compound 15 is (E)-5-Chloro-3-(4′-(dimethylamino)benzylidene)indolin-2-one which may be prepared by Method B in 84% yield, mp 257-260° C. ¹H NMR (400 MHz, DMSO-d₆)™ 10.59 (s, 1H, NH-1), 7.70 (s, 1H, H-4), 7.63 (d, J=7.88 Hz, 1H, H-2′,6′), 7.59 (s, 1H, H-vinyl), 7.21 (d, J=7.88 Hz, 1H, H-3′), 6.82-6.87 (m, 3H, 5,7,5′), 3.04 (s, 6H, N(CH₃)₂); NOE between H-vinyl and H-2′,6′. ¹³C NMR (400 MHz, DMSO-d₆)™ 169.9 (CO), 152.5 (C), 141.5 (C), 140.1 (CH), 133.0 (CH), 128.8 (CH), 125.5 (C), 124.4 (CH), 121.8 (C), 121.7 (C), 121.4 (C), 112.3 (CH), 111.8 (CH), 40.8 (N(CH₃)₂); HRMS Calcd. for C₁₇H₁₅ClN₂O: 298.0873. Found: 298.0881.

As used herein, compound 16 is (E)-5-Bromo-3-(3′,5′-dibromo-4-hydroxybenzylidene)indolin-2-one which may be prepared by Method A in 78% yield, mp 148-150° C. ¹H NMR (500 MHz, DMSO-d₆)™ 10.70 (s, 1H, NH-1), 7.87 (s, 2H, H-2′,6′), 7.54 (s, 1H, H-vinyl), 7.50 (s, 1H, H-4), 7.36 (d, J=8.60 Hz, 1H, H-7), 6.80 (d, J=8.60 Hz, 1H, H-6); NOE effect between H-vinyl and H-2′,6′. ¹³C NMR (500 MHz, DMSO-d₆)™ 168.9 (CO), 152.7 (C), 142.3 (C), 135.3 (CH), 133.8 (CH), 129.5 (CH), 128.9 (C), 127.5 (C), 124.4 (C), 120.9 (C), 111.2 (CH), 110.8 (C), 110.1 (CH); HRMS Calcd. for C₁₅H₈Br₃NO₂: 470.9408. Found: 470.9419.

As used herein, compound 17 is (Z)-5-Bromo-3-(3′,5′-dibromobenzylidene)indolin-2-one which may be prepared by Method B in 81% yield mp 282-284° C. ¹H NMR (500 MHz, DMSO-d₆)™ 10.80 (brs, 1H, NH), 8.59 (s, 2H, H-2′,6′), 7.87-7.94 (m, 3H, H-4′, H-vinyl, H-4), 7.35-7.38 (m, 1H, H-6), 6.76 (d, J=8.02 Hz); NOE for H-vinvyl; and H-2′,6′. HRMS Calcd. for C₁₅H₈Br₃NO: 470.9408. Found: 470.9416.

As used herein, compound 18 is (Z)-5-Bromo-3-(3′, 4′, 5′-trimethoxybenzylidene)indolin-2-one which may be prepared by Method B in 80% yield, mp 250-252° C. ¹H NMR (500 MHz, DMSO-d₆)™ 10.68 (brs, 1H, NH), 8.01 (s, 2H, H-2,6′), 7.89 (s, 1H, H-vinyl), 7.87 (s, 1H, H-4), 7.31 (dd, J=1.1, 9.1 Hz, 1H, H-6), 6.76 (d, J=8.6 Hz, 1H, H-7), 3.81 (s, 6H, 2×OCH₃), 3.72 (s, 3H, OCH₃); NOE effect between H-vinyl and H-4. ¹³C NMR (500 MHz, DMSO-d₆)™ 168.9 (CO), 152.8 (C), 140.5 (C), 139.9 (CH), 131.2 (CH), 130.0 (C), 128.2 (C), 124.5 (C), 122.7 (CH), 113.5 (C), 111.7 (CH), 110.9 (CH), 60.7 (CH₃), 56.4 (CH₃); HRMS Calcd. for C₁₈H₁₆BrNO₄: 375.0232. Found: 375.0237.

As used herein, compound 19 is (E)-5-Bromo-3-(4′-methoxybenzylidene)indolin-2-one which may be prepared by Method B in 83% yield, mp 220-222° C. ¹H NMR (500 MHz, DMSO-d₆)™ 10.68 (brs, 1H, NH), 7.66-7.68 (m, 3H, H-4, H-2′,6′), 7.62 (s, 1H, H-vinyl), 7.36 (dd, J=1.72, 8.02 Hz, 1H, H-6), 7.08 (d, J=8.59 Hz, 2H, H-3′,5′), 6.81 (d, J=8.59 Hz, 1H, H-7), 3.82 (s, 3H, OCH₃); NOE for H-vinyl H-2′,6′, HRMS Calcd. for C₁₆H₁₂BrNO₂: 329.0051. Found: 329.0059.

As used herein, compound 20 is (E)-5-Bromo-3-(4′-(dimethylamino)benzylidene) indolin-2-one which may be prepared by Method B in 81% yield, mp 238-240° C. ¹H NMR (500 MHz, DMSO-d₆)™ 10.55 (brs, 1H, NH), 7.80 (d, J=6.8 Hz, 1H, H-4), 7.59 (d, J=9.1 Hz, 2H, H-2′,6′), 7.54 (s, 1H, H-vinyl), 7.20-7.32 (dd, J=1.72, 8.05 Hz, 1H, H-6), 6.76-6.83 (m, 3H, H-3′,5′, H-7), 3.00 (s, 6H, N(CH₃)₂); NOE between H-vinyl and H-2′,6′, HRMS Calcd. for C₁₇H₁₅BrN₂O: 342.0368. Found: 342.0376.

As used herein, compound 21 is (Z)-3-(3′,5′-Dibromo-4-hydroxybenzylidene)-5-nitroindolin-2-one which may be prepared by Method A in 78% yield, mp 325-327° C. ¹H NMR (500 MHz, DMSO-d₆)™ 11.30 (s, 1H, NH-1), 8.77 (s, 2H, H-2′, 6′), 8.55 (s, 1H, H-vinyl), 8.10 (d, J=8.60 Hz, 1H, H-4), 7.36 (d, J=8.90 Hz, 1H, H-7), 6.94 (d, J=8.90 Hz, 1H. H-6); NOE effect for H-vinyl H-4; HRMS Calcd. for C₁₅H₈Br₂N₂O₃: 421.8902. Found: 421.8918.

As used herein, compound 22 is (Z)-3-(3′,5′-Dibromobenzylidene)-5-nitroindolin-2-one which may be prepared by Method B in 80% yield, mp 304-306° C. ¹H NMR (500 MHz, DMSO-d₆)™ 11.38 (brs, 1H, NH), 8.61-8.63 (m, 3H, H-2′,6′, H-4′), 8.12-8.18 (m, 2H, vinyl-H, H-4), 7.92-7.96 (m, 1H, H-6), 6.98 (d, J=9.16 Hz, H-7); NOE effect between H-vinyl and H-4; HRMS Calcd. for C₁₅H₈Br₂N₂O₄: 437.8851. Found: 437.8861.

As used herein, compound 23 is (Z)-3-(3′, 4′, 5′-Trimethoxybenzylidene)-5-nitroindolin-2-one which may be prepared by Method B in 83% yield, mp 300-302° C. ¹H NMR (500 MHz, DMSO-d₆)™ 11.27 (brs, 1H, NH), 8.63 (s, 1H, H-4), 8.14-8.12 (m, 2H, 1H-vinyl, H-6), 8.07 (s, 2H, H-2′,6′), 7.00 (d, J=8.6 Hz, 1H, H-7), 3.83 (s, 6H, 2×OCH₃), 3.74 (s, 3H, OCH₃); NOE between H-vinyl and H-4; ¹³C NMR (500 MHz, DMSO-d₆)™ 169.5 (CO), 168.0 (C), 153.5 (C), 152.8 (C), 148.0 (C), 141.7 (CH), 140.1 (CH), 126.8 (CH), 125.4 (CH), 118.3 (CH), 115.6 (CH), 111.2 (CH), 110.5 (CH), 109.8 (CH), 108.1 (CH), 60.7 (CH₃), 56.5 (CH₃), 56.3 (CH₃); HRMS Calcd. for C₁₈H₁₆N₂O₆: 356.1008. Found: 356.1014.

As used herein, compound 24 is (E)-3-(2′,6′-Dichlorobenzylidene)-5-nitroindolin-2-one which may be prepared by Method B in 85% yield, mp 282-284° C. ¹H NMR (500 MHz, DMSO-d₆)™ 11.46 (brs, 1H, NH), 8.15 (dd, J=2.2, 9.1 Hz, 1H, H-4), 7.66-7.70 (m, 3H, H-3′,5′, H-vinyl), 7.57-7.60 (m, 1H, H-4′), 7.26 (d, J=2.29 Hz, 1H, H-6), 7.04 (d, J=8.59 Hz, 1H, H-7); ¹³C NMR (500 MHz, DMSO-d₆)™ 168.1 (CO), 149.1 (C), 142.4 (C), 133.6 (C), 132.4 (CH), 132.4 (C), 132.0 (C), 130.5 (C), 129.4 (CH), 127.9 (CH), 121.4 (C), 118.4 (C), 111.0 (CH); HRMS Calcd. for C₁₅H₈Cl₂N₂O₃: 333.9912. Found: 333.9923.

As used herein, compound 25 is (Z)-3-Benzylidene-5-nitroindolin-2-one which may be prepared by Method B in 80% yield, mp 220-222° C. ¹H NMR (500 MHz, DMSO-d₆)™ 11.32 (brs, 1H, NH), 8.67 (d, J=2.29 Hz, 1H, H-4), 8.39-8.40 (m, 2H, H-2′,6′), 8.20 (s, 1H, H-vinyl), 8.14 (dd, J=2.3, 8.6 Hz, 1H, H-6), 7.47-7.48 (m, 3H, H-3′,4′,5′), 6.97 (d, J=8.6 Hz, 1H, H-7); NOE effect between H-vinyl and H-4; HRMS Calcd. for C₁₅H₁₀N₂O₃: 266.0691. Found: 266.0699.

As used herein, compound 26 is (E)-5-Nitro-3-(E)-3-phenylallylidene)indolin-2-one which may be prepared by Method B in 81% yield, mp 302-304° C. ¹H NMR (500 MHz, DMSO-d₆)™ 11.22 (brs, 1H, NH), 8.51 (s, 1H, H-4), 8.37-8.42 (m, 1H, H-9), 8.09-8.11 (m, 1H, H_{b}), 7.98 (dd, J=2.29, 11.4 Hz, 1H, H-7), 7.59-7.60 (m, 2H, 2′,6′-H), 7.36-7.47 (m, 3H, 3,4′,5′-H), 7.26 (d, J=16.04 Hz, 1H, H_{c}), 6.97 (dd, J=1.72, 8.6 Hz, 1H, H_{b}); NOE effect between H-vinyl and 2′,6′-H; ¹³C NMR (500 MHz, DMSO-d₆)™ 168.9 (CO), 146.8 (C), 145.4 (CH), 142.5 (CH), 140.0 (CH), 136.3 (C), 130.5 (CH), 129.7 (CH), 129.5 (C), 128.8 (C), 128.2 (CH), 125.7 (CH), 124.8 (C), 124.1 (CH), 123.6 (C), 116.2 (CH), 110.0 (CH); HRMS Calcd. for C₁₇H₁₂N₂O₃: 292.0848. Found: 292.0857.

As used herein, compound 27 is (E)-5-Nitro-3-(2′-nitrophenyl)allylidene) indolin-2-one which may be prepared by Method B in 83% yield, mp 325-327° C. ¹H NMR (500 MHz, DMSO-d₆)™ 11.26 (brs, 1H, NH), 8.57 (s, 1H, H-4), 8.35-8.40 (m, 1H, Hₐ), 8.08-8.13 (m, 2H, H-3′, H-6), 8.02 (d, J=7.45 Hz, 1H, H-7), 7.86 (d, J=7.45 Hz, 1H, H-6′), 7.78 (t, J=8.02 Hz, 1H, H-5′), 7.62 (t, J=7.45 Hz, 1H, H-4′), 7.45 (d, J=14.89 Hz, 1H, H_{c}), 6.96 (d, J=8.02 Hz, 1H, H_{b}); NOE effect for H-vinyl and H-2′,6′; ¹³C NMR (500 MHz, DMSO-d₆)™168.7 (CO), 148.9 (C), 147.2 (C), 143.0 (C), 138.5 (CH), 138.1 (CH), 134.0 (CH), 130.6 (CH), 128.8 (CH), 128.5 (CH), 126.0 (CH), 125.0 (CH), 116.6 (CH), 110.1 (CH); HRMS Calcd. for C₁₇H₁₁N₃O₅: 337.0699. Found: 337.0689.

As used herein, compound 28 is (Z)-3-(4′-Methylbenzylidene)-5-nitroindolin-2-one which may be prepared by Method B in 80% yield, mp 263-265° C. ¹H NMR (500 MHz, DMSO-d₆)™ 11.29 (brs, 1H, NH), 8.66 (d, J=1.72 Hz, 1H, H-4), 8.35 (d, J=8.02 Hz, 2H, H-2′,6′), 8.16 (s, 1H, H-vinyl), 8.12 (dd, J=2.2, 9.7 Hz, 1H, H-6), 7.29 (d, J=8.59 Hz, 2H, H-3′,5′), 6.97 (d, J=8.5 Hz, 1H, H-7), 2.35 (s, 1H, CH₃); NOE between H-vinyl and H-4; HRMS Calcd. for C₁₆H₁₂N₂O₃: 294.0879. Found: 294.0885.

As used herein, compound 29 is (Z)-3-(4′-(Dimethylamino)benzylidene)-5-nitroindolin-2-one which may be prepared by Method B in 81% yield, mp 295-297° C. ¹H NMR (500 MHz, DMSO-d₆)™ 11.13 (brs, 1H, NH), 8.54 (d, J=2.29 Hz, 1H, H-4), 8.48 (d, J=9.16 Hz, 2H, H-2′,6′), 8.02 (dd, J=2.3, 8.59 Hz, 1H, H-6), 7.98 (s, 1H, H-vinyl), 6.93 (d, J=8.52 Hz, 1H, H-7), 6.76 (d, J=8.89 Hz, 2H, H-3′,5′), 3.03 (s, 6H, N(CH₃)₂); NOE effect between H-vinyl and H-4. ¹³C NMR (500 MHz, DMSO-d₆)™ 168.3 (CO), 153.0 (C), 145.1 (C), 142.4 (CH), 142.2 (C), 136.2 (CH), 127.6 (C), 123.6 (CH), 122.2 (C), 117.6 (C), 114.4 (CH), 111.6 (CH), 109.2 (CH), 40.2 (CH₃), 40.1 (CH₃); HRMS Calcd. for C₁₇H₁₅N₃O₃: 309.1113. Found: 309.1120.

As used herein, compound 30 is (E)-3-(3′,5′-Dibromo-4′-hydroxybenzylidene)-5-fluoro-indolin-2-one which may be prepared by Method A in 70% yield, mp 173-175° C. ¹H NMR (500 MHz, DMSO-d₆)™ 10.58 (s, 1H, NH-1), 7.87 (s, 2H, H-2′, 6′), 7.51 (s, 1H, H-vinyl), 7.17 (d, J=9.20 Hz, 1H, H-4), 7.07 (dt, J=2.90, 9.20 Hz, 1H, H-7), 6.81 (dd, J=4.8 0, 8.60 Hz, 1H, H-6); NOE effect between H-vinyl and H-2,6′; ¹³C NMR (500 MHz, DMSO-d₆)™ 168.9 (CO), 158.5 (C), 152.7 (C), 139.9 (C), 135.2 (CH), 133.8 (CH), 128.7 (C), 127.8 (C), 122.1 (C), 117.1 (CH), 112.3 (C), 111.4 (CH), 109.5 (CH), HRMS Calcd. for C₁₅H₈Br₃NO₂: 410.8906. Found: 410.8913.

As used herein, compound 31 is (E)-3-(3′,5′-Dibromobenzylidene)-5-fluoroindolin-2-one which may be prepared by Method B in 88% yield, mp 290-293° C. ¹H NMR (500 MHz, DMSO-d₆)™ 10.66 (s, 1H, NH-1), 7.94 (s, 1H, H-4′), 7.88 (s, 2H, H-2′, 6′), 7.58 (s, 1H, H-vinyl), 7.07-7.11 (m, 2H, H-6,4), 7.21 (d, J=8.85 Hz, 1H, H-6), 6.83-6.85 (m, 1H, H-7); NOE effect between H-vinyl and H-2′,6′; HRMS Calcd. for C₁₅H₈Br₂FNO: 394.8957. Found: 394.8969.

As used herein, compound 32 is (Z)-5-Fluoro-3-(3′, 4′, 5′-trimethoxybenzylidene)indolin-2-one which may be prepared by Method B in 87% yield, mp 194-196° C. ¹H NMR (400 MHz, DMSO-d₆)™ 10.49 (s, 1H, NH-1), 7.91 (s, 2H, H-2′,6′), 7.73 (s, 1H, H-vinyl), 7.47 (dd, J=2.4, 8.7 Hz, 1H, H-4), 6.75-6.89 (m, 1H, H-7), 6.69-6.73 (m, 1H, H-6), 3.74 (s, 6H, 2×OCH₃), 3.64 (s, 3H, OCH₃); NOE effect between H-vinyl and H-4; ¹³C NMR (400 MHz, DMSO-d₆)™ 168.1 (CO), 159.9 (C), 153.1 (C), 140.9 (C), 139.9 (CH), 137.4 (C), 130.1 (C), 127.6 (C), 126.0 (C), 115.7 (CH), 111.2 (CH), 110.9 (CH), 108.0 (CH), 61.0 (OCH₃), 56.9 (OCH₃); HRMS Calcd. for C₁₈H₁₆FNO₄: 329.1063. Found: 329.1073.

As used herein, compound 33 is (E)-3-Benzylidene-5-fluoroindolin-2-one which may be prepared by Method B in 86% yield, mp 195-198° C. ¹H NMR (500 MHz, DMSO-d₆)™10.61 (s, 1H, NH-1), 7.65-7.68 (m, 3H, H-2′,6′, vinyl), 7.48-7.53 (m, 3H, H-3′, 5′,4), 7.47 (d, J=8.60 Hz, 1H, H-6), 7.06 (t, J=8.75 Hz, 1H, H-4′), 6.84 (d, J=8.60 Hz, 1H, H-7); NOE between H-vinyl and H-2′,6′; ¹³C NMR (500 MHz, DMSO-d₆)™ 169.0 (CO), 139.8 (C), 138.0 (CH), 134.5 (C), 130.5 (CH), 129.7 (CH), 129.4 (CH), 117.1 (CH), 111.3 (CH), 109.7 (CH); HRMS Calcd. for C₁₅H₁₀FNO: 239.0746. Found: 239.0754.

As used herein, compound 34 is (Z)-5-Acetyl-3-(3′,5′-dibromo-4′-hydroxybenzylidene)indolin-2-one which may be prepared by Method A in 74% yield, mp 286-289° C. ¹H NMR (500 MHz, DMSO-d₆)™ ¹H NMR (500 MHz, DMSO-d₆) d 11.03 (s, 1H, NH-1), 10.72 (s, 1H, OH), 8.80 (s, 2H, H-2′, 6′), 8.28 (s, 1H, H-vinyl), 7.91 (d, J=6.85 Hz, 1H, H-4), 7.83 (d, J=8.05 Hz, 1H, H-7), 6.88 (dd, J=8.05, 4.05 Hz, 1H, H-6), 2.47 (s, 3H, CH₃); NOE between H-vinyl and H-4; ¹³C NMR (500 MHz, DMSO-d₆)™ 197 (CO), 168.1 (CO), 153.4 (C), 145.0 (C), 136.9 (CH), 136.1 (CH), 131.1 (C), 130.4 (CH), 129.0 (C), 125.4 (C), 120.4 (CH), 111.6 (C), 109.5 (CH), 27.0 (CH₃); HRMS Calcd. for C₁₇H₁₁Br₂NO: 434.9106. Found: 434.9120.

As used herein, compound 35 is (Z)-3-(1H-Pyrrol-2′-yl)methylene)indolin-2-one which may be prepared by Method B in 86% yield, mp194-196° C. The ¹H NMR was identical to that reported previously [5]. ¹H NMR (500 MHz, DMSO-d₆)™ ¹H 13.35 (s, 1H, NH-1′), 10.87 (s, 1H, NH-1), 7.70 (s, 1H, H-vinyl), 7.59 (d, J=7.45 Hz, 1H, H-4), 7.31 (s, 1H, H-5′), 7.11 (t, J=7.45 Hz, 1H, H-6), 6.96 (t, J=7.45 Hz, 1H, H-5), 6.86 (d, J=7.45 Hz, 1H, H-7), 6.80 (t, J=1.69 Hz, 1H, H-3′), 6.82-6.84 (m, 1H, H-4′); NOE between H-vinyl and H-3′; ¹³C NMR (500 MHz, DMSO-d₆)™ 169.7 (CO), 139.5 (C), 130.1 (C), 127.4 (CH), 126.8 (CH), 126.1 (CH), 125.7 (C), 121.7 (CH), 120.8 (CH), 119.0 (CH), 117.3 (C), 111.9 (CH), 110.0 (CH); HRMS Calcd. for C₁₃H₁₀Br₂N₂O: 367.9160. Found: 367.9176.

As used herein, compound 36 is (Z)-5-Chloro-(1H-pyrrol-2′-yl)methylene)indoline-2-one which may be prepared by Method B in 90% yield, mp 260-262° C. ¹H NMR spectra was identical to that reported previously [5]. ¹H NMR spectrum (400 MHz, DMSO-d₆)™ 13.30 (s, 1H, NH-1′), 10.14 (s, 1H, NH-1), 7.85 (s, 1H, H-vinyl), 7.74 (d, J=2.4 Hz, 1H, H-4), 7.38-7.41 (m, 1H, H-5′), 7.16 (dd, J=2.4, 8.05 Hz, 1H, H-6), 6.90 (d, J=8.05 Hz, 1H, H-7), 6.86 (dd, J=1.59, 3.44 Hz, 1H, H-3′), 6.36-6.39 (m, 1H, H-4′); NOE effect between H-vinyl and H-3′; ¹³C NMR (400 MHz, DMSO-d₆)™ 169.9 (CO), 138.3 (C), 130.3 (C), 128.8 (CH), 128.0 (C), 127.4 (CH), 126.9 (CH), 126.4 (C), 122.1 (CH), 119.3 (CH), 116.4 (C), 112.6 (CH), 111.6 (CH); HRMS Calcd. for C₁₃H₉ClN₂O: 244.0403. Found: 244.0424.

As used herein, compound 37 is (Z)-5-Bromo (3-(1H-pyrrol-2′-yl)methylene)indolin-2-one which may be prepared by Method B in 83% yield, mp 262-265° C. ¹H NMR (500 MHz, DMSO-d₆)™ 10.98 (brs, 1H, NH), 7.86 (s, 1H, H-vinyl), 7.84 (d, J=1.72 Hz, H-4), 7.36-7.38 (m, 1H, H-5′), 7.24 (dd, J=2.29, 8.0 Hz, 1H, H-6), 6.78-6.81 (m, 2H, H-7, H-3′), 6.34-6.35 (m, 1H, H-4′); NOE between H-vinyl and H-3′; ¹³C NMR (500 MHz, DMSO-d₆)™169.4 (CO), 138.3 (C), 130.0 (C), 129.4 (CH), 128.5 (CH), 128.1 (C), 127.1 (CH), 121.9 (CH), 121.7 (CH), 115.8 (C), 113.8 (C), 112.3 (CH), 111.8 (CH); HRMS Calcd. for C₁₃H₉BrN₂O: 287.9898. Found: 287.9890.

As used herein, compound 38 is (Z)-5-Acetyl-3-(1H-pyrrol-2′-yl)methylene)indolin-2-one which may be prepared by Method B in 82% yield, mp 132-134° C. ¹H NMR (500 MHz, DMSO-d₆)™ 11.23 (brs, 1H, NH), 8.26 (d, J=1.72 Hz, 1H, H-4), 7.95 (s, 1H, H-vinyl), 7.79 (dd, J=1.72, 8.6 Hz, 1H, H-6), 7.35-7.37 (m, 1H, H-5′), 6.95 (d, J=8.02 Hz, 1H, H-7), 6.85-6.88 (m, 1H, H-3′), 6.35-6.36 (m, 1H, H-4′), 2.54 (s, 3H, CH₃); NOE between H-vinyl and H-3′; ¹³C NMR (500 MHz, DMSO-d₆)™ 197.3 (CO), 170.1 (CO), 143.2 (C), 131.2 (C), 130.1 (CH), 128.4 (CH), 128.3 (CH), 127.0 (CH), 125.8 (C), 121.9 (CH), 119.4 (CH), 116.0 (CH), 112.3 (CH), 109.7 (CH), 27.0 (CH₃); HRMS Calcd for C₁₅H₁₂N₂O₂: 252.0899. Found: 252.0905.

As used herein, compound 39 is (Z)-5-Nitro-3-(1H-pyrrol-2-yl)methylene)indolin-2-one which may be prepared by Method B in 80% yield, mp 308-310° C. The ¹H NMR spectrum was identical to that previously reported [5]. ¹H NMR spectrum (500 MHz, DMSO-d₆)™ 11.50 (brs, 1H, NH), 8.56 (d, J=2.29 Hz, 1H, H-4), 8.13 (s, 1H, H-vinyl), 8.05 (dd, J=2.29, 8.35 Hz, 1H, H-6), 7.42-7.43 (m, 1H, H-5′), 7.03 (d, J=8.59 Hz, 1H, H-7), 6.92-6.93 (m, 1H, H-3′), 6.39-6.40 (m, 1H, H-4′); NOE between H-vinyl and H-3′; ¹³C NMR (500 MHz, DMSO-d₆)™ 170.1 (CO), 144.5 (C), 142.7 (C), 130.15 (CH), 130.1 (C), 128.2 (CH), 126.6 (C), 123.4 (CH), 123.2 (CH), 114.6 (CH), 112.7 (CH), 109.9 (CH); HRMS Calcd. for C₁₃H₉N₃O₃: 255.0644. Found: 255.0712.

As used herein, compound 40 is (Z)-5-Fluoro-(pyrrol-2′-yl)methylene)indoline-2-one which may be prepared by Method B in 85% yield, mp 240-243° C. ¹H NMR (400 MHz, DMSO-d₆)™ 13.35 (s, 1H, NH-1′), 10.90 (s, 1H, NH-1), 7.85 (s, 1H, H-vinyl), 7.70 (d, J=2.5 Hz, 1H, H-4), 7.38-7.40 (m, 1H, H-5′), 7.10 (dd, J=2.5, 8.05 Hz, 1H, H-6), 6.95 (d, J=8.05 Hz, 1H, H-7), 6.85 (dd, J=1.60, 3.45 Hz, 1H, H-3′), 6.37-6.39 (m, 1H, H-4′); NOE between H-vinyl and H-3′; ¹³C NMR (400 MHz, DMSO-d₆)™ 170.1 (CO), 160.1 (C), 135.9 (C), 130.3 (C), 128.6 (CH), 127.8 (CH), 121.9 (CH), 117.2 (C), 113.9 (C), 113.6 (CH), 112.6 (CH), 111.0 (C), 106.5 (CH); HRMS Calcd. for C₁₃H₉FN₂O: 228.0699. Found: 228.0705.

As used herein, compound 41 is (Z)-3-(Thien-2′-ylmethylene)indolin-2-one which may be prepared by Method B; brown solid; 80% yield; mp 210° C. (Lit. [8] 210° C.). ¹H NMR (500 MHz, DMSO-d₆)™ 10.58 (s, 1H, NH-1), 8.13 (d, J=8.05 Hz, 1H, H-4), 7.96 (s, H, H-vinyl), 7.75-7.79 (m, 2H, H-2′,4′), 7.25-7.28 (m, 2H, H-5,6), 7.00 (t, J=7.45 Hz, 1H, H-3′), 6.89 (d, J=6.25 Hz, 1H, H-7); NOE between H-vinyl and H-3′; ¹³C NMR (500 MHz, DMSO-d₆)™ 169.7 (CO), 143.1 (C), 137.6 (CH), 136.6 (C), 132.5 (CH), 130.4 (CH), 129.1 (CH), 127.6 (CH), 123.9 (C), 123.7 (CH), 121.7 (CH), 121.4 (C), 110.5 (CH); HRMS Calcd. for C₁₃H₈NOS: 226.0327. Found: 226.0337.

As used herein, compound 42 is (Z)-5-Nitro-3-(thien-2′-ylmethylene) indolin-2-one which may be prepared by Method B in 81% yield, mp 291-293° C. ¹H NMR (500 MHz, DMSO-d₆)™ 11.28 (brs, 1H, NH), 8.63 (d J=2.29 Hz, H-4), 8.52 (s, 1H, H-vinyl), 8.10 (dt, J=2.29, 8.55 Hz, 1H, H-6), 7.97-7.98 (m, 2H, H-3′,5′), 7.24-7.26 (m, 1H, H-4′), 6.99 (dd, J=8.55 Hz, 1H, H-7); NOE between H-vinyl and H-3′; ¹³C NMR (500 MHz, DMSO-d₆)™ 168.0 (CO), 146.2 (C), 142.4 (C), 139.8 (CH), 137.6 (C), 136.7 (CH), 132.5 (CH), 128.4 (CH), 125.7 (C), 125.2 (CH), 119.7 (C), 115.7 (CH), 109.9 (CH); HRMS Calcd for C₁₃H₈N₂O₃S: 272.0256. Found: 272 0268.

As used herein, compound 43 is (Z)-5-Bromo-3-(thien-2′-ylmethylene)indolin-2-one which may be prepared by Method B in 86% yield, mp 265-266° C. ¹H NMR (500 MHz, DMSO-d₆)™ 10.69 (brs, 1H, NH), 8.22 (s, 1H, H-vinyl), 7.89-7.90 (m, 3H, H-4, H-3′,5′), 7.31 (dd, J=2.29, 8.05 Hz, 1H, H-6), 7.21-7.22 (m, 1H, H-4′), 6.77 (d, J=8.02 Hz, 1H, H-7); ¹³C NMR (500 MHz, DMSO-d₆)™ 167.4 (C), 139.9 (C), 138.8 (CH), 137.7 (C), 135.7 (CH), 131.0 (CH), 130.6 (CH), 128.2 (CH), 122.7 (CH), 120.9 (C), 113.5 (C), 111.8 (CH); HRMS Calcd. for C₁₃H₈BrNOS: 304.9510. Found: 304.9518.

As used herein, compound 44 is (Z)-5-Fluoro-3-(thien-2′-ylmethylene)indolin-2-one which may be prepared by Method B in 80% yield, 188-190° C. ¹H NMR (500 MHz, DMSO-d₆)™ 10.58 (s, 1H, NH-1), 8.15 (s, 1H, H-vinyl), 7.90-7.92 (m, 2H, H-2′,4′), 7.56 (d, J=9.15 Hz, 1H, H-4), 6.99 (t, J=4.0 Hz, 1H, H-3′), 6.98 (dt, J=1.20, 7.45 Hz, 1H, H-6), 6.79 (d, J=7.45 Hz, 1H, H-7); NOE between H-vinyl and H-3′; ¹³C NMR (500 MHz, DMSO-d₆)™ 167.8 (CO), 159.3 (C), 138.6 (CH), 137.7 (C), 137.1 (C), 135.5 (CH), 130.2 (CH), 128.2 (CH), 121.8 (C), 115.2 (CH), 110.7 (CH), 107.4 (CH); HRMS Calcd. for C₁₃H₈FNO₂: 229.0539. Found: 229.0548.

As used herein, compound 45 is (E)-3-(Furan-2′-ylmethylene)indoline-2-one which may be prepared by Method C; red solid; yield 85%; mp 178-181° C. (lit [8], mp 183). ¹H NMR (500 MHz, DMSO-d₆)™ 10.55 (s, 1H, NH-1), 8.35 (d, J=7.45 Hz, 1H, H-4), 8.14 (d, J=1.85 Hz, 1H, H-5′), 7.31 (s, 1H, H-vinyl), 7.21-7.24 (m, 2H, H-6,3′), 6.99 (dt, J=1.15, 7.45 Hz, 1H, H-5), 6.85 (d, J=7.49 Hz, 1H, H-7), 6.78 (dd, J=1.85, 3.45 Hz, 1H, H-4′); NOE effect between H-vinyl and H-3′; ¹³C NMR (500 MHz, DMSO-d₆)™ 169.6 (CO), 151.0 (C), 147.9 (CH), for f(CH), 114.1 (CH), 110.3 (CH).

These compounds were analyzed for their neuroprotective activity in vitro. Granule neuron cultures were obtained from dissociated cerebella of 7-8 day old Wistar rats as known to one of ordinary skill in the art. Cells were plated in Basal Eagle's Medium with Earles salts (BME) supplemented with 10% fetal calf serum (FCS), 25 mM KCl, 2 mM glutamine (Invitrogen), and 100 gg/ml gentamycin on dishes (Nunc) coated with poly-L-lysine in 24-well dishes at a density 1×10⁶ cells/well. Cytosine arabinofuranoside (10 μM) was added to the culture medium 18-22 h after plating to prevent replication of non-neuronal cells that yields highly pure culture of granule neurons. The neuronal cultures were maintained for 7-8 days prior to treatments.

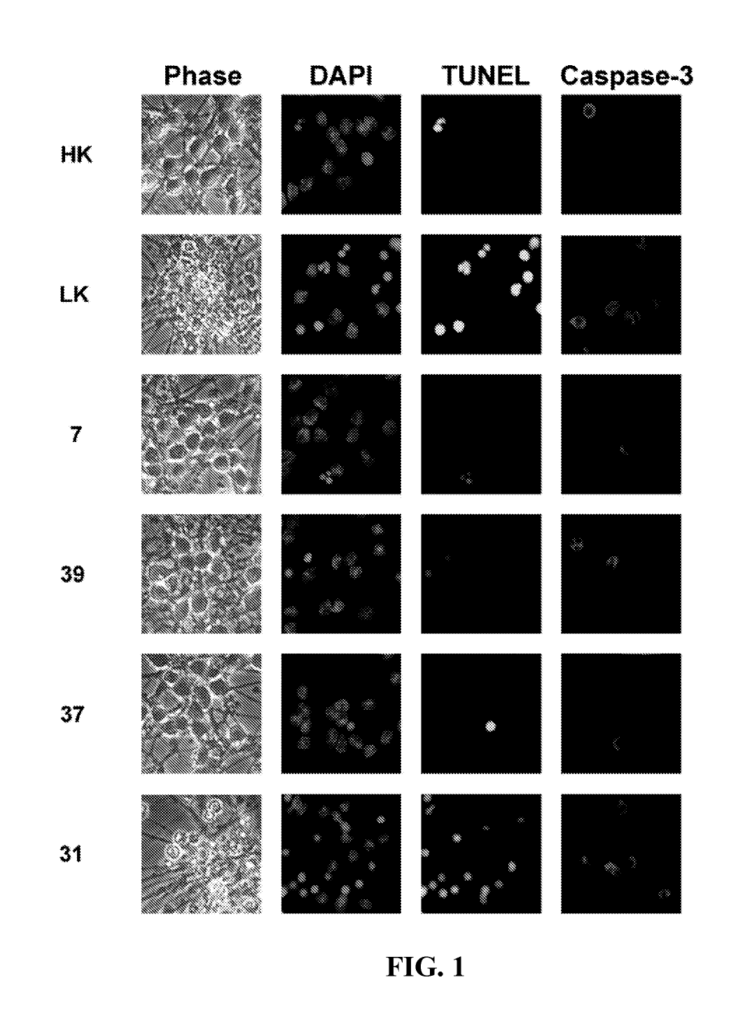

When switched from HK medium (containing 25 μM KCl) in which they are normally maintained in vitro, to LK medium (containing 5 μM KCl), these neurons die by apoptosis, killing about 50% of the neurons within 24 hours, as shown in FIG. 1 and known to one of ordinary skill in the art. This well-known and accepted model of neuronal apoptosis has been used widely used to understand the molecular mechanisms underlying neurodegeneration and to identify biological and chemical agents with neuroprotective efficacy. For this, the cells were rinsed once and then maintained in low K+ medium (serum-free BME medium, 5 mM KCl; referred to as LK), or in the case of control cultures, in high K+ medium (serum-free BME medium, supplemented with 20 mM KCl; referred to as HK). Each compound was tested at three different concentrations of 1 μM, 5 μM, and 25 μM. The 25 μM concentration was included to evaluate neurotoxicity. For treatments, the chemical compounds (dissolved in dimethylsulfoxide) were added directly to LK medium at the time of the switch from HK at concentrations of 1 μM, 5 μM, or 25 μM. Viability was assessed 24 hours later. Each compound was tested in duplicate (at each of the concentrations) and the experiment repeated at least 3 times. Although all compounds were solubilized in dimethylsulfoxide (DMSO), the amount of DMSO in the cultures never exceeded 0.1% (v/v). DMSO has no effect on neuronal viability when used at dilutions of over 1:1000.

The viability status of neuronal cultures treated with HK, LK, or LK medium supplemented with various compounds was evaluated by phase contrast microscopy and quantified by staining cell nuclei with 4′,6′-diamidino2-phenylindole hydrochloride (DAPI). Briefly, the cells were fixed in 4% paraformaldehyde for 20 minutes at 4° C. After washing in phosphate buffered saline, DAPI (1 gg/ml in phosphate buffered saline) was added for 15 min at room-temperature and viewed under ultraviolet light (260 nm). Cells with condensed or fragmented nuclei were scored as dead. Viability has been expressed as percent of control cultures, which were switched to HK medium. Statistical analysis was performed using an unpaired, two-tailed Student's t test, compared to mean neuronal survival of control cultures receiving LK treatment and the quantitative data are presented in Tables 1 and 2 for each compound.

FIG. 1 is a photograph illustrating the qualitative neuroprotective effects of Compound 7 (5 μm), Compound 31 (1 μm), Compound 37 (25 μm) and Compound 39 (25 μm) are illustrated presented in the FIGURE. From left to right columns show phase-contrast micrographs of neuronal morphology, DAPI staining of nuclei showing fragmented nuclei, TUNEL staining also showing fragmented DNA, and active caspase-3 immunoreactivity highly indicative of apoptosis. DAPI and TUNEL panels are taken from the same fields. In contrast to Compounds 7, 37, and 39, Compound 31 illustrates greater TUNEL and active caspase-3 immunoreactivity reflecting its comparatively lack of neuroprotective activity.

The TUNEL assay of neuronal cultures was performed 24 h after treatment of the cultures using DEADEND™ Fluorometric TUNEL System from Promega (Madison, Wis.) according to the manufacturer's instructions. For immunocytochemical analysis of active caspase 3, neuronal cultures cells were fixed and treated with 0.2% Triton for 5 minutes. After blocking with PBS containing 5% BSA and 5% goat serum in PBS for 30 minutes, the coverslips were incubated with the active capase-3 primary antibody overnight at 4° C. After three washes with phosphate-buffered saline (PBS), the cells were incubated with secondary antibodies for 45 minutes at 25° C. after which the cells were washed with PBS. To visualize nuclei, cells were stained with DAPI for 15 minutes at 25° C.

When cerebellar granule neuron cultures are switched to LK medium in the absence of any neuroprotective agent about 50% of the cells die within 24 hours. Several of the compounds tested displayed a significant level of protection against LK-mediated neuronal death. Among these, Compounds 7, 16, 34, 37, 39, and 45 were deemed to be highly neuroprotective (>90% survival in LK medium) at least one of the three doses. In the case of Compound 34, however, while exhibiting impressive neuroprotection at 1 μM and 5 μM concentrations, the drug is highly toxic at 25 μM. Its high level of toxicity renders it unsuitable for consideration as a potential therapeutic agent. The efficacy of Compound 16 which is highly protective at the two lower concentrations could not be evaluated at 25 μM because it was not completely soluble at this dose in the culture medium. In contrast, Compounds 4, 7, 14, and 39 show solid neuroprotective activity providing at least 80% survival at all three concentrations utilized in this study. A number of compounds displayed protection of over 70% survival at all three concentrations at which they were tested, in particular compounds 6, 9, 21, 22, 26, 36, and 45.

The substituent effects on the neuroprotective ability of 3-(3′,5′-dibromo-4′-hydroxybenzyli-dene)indolin-2-one were examined. As shown in Table 1, compound 1 is moderately effective and not toxic. Further, the importance of an acidic phenol was determined by observing that the absence of a 4′-phenolic group as in 3′,5′-dibromo (Compound 2) and 3′,4′,5′-trimethoxy derivatives (Compound 3) are slightly toxic at 5 and 25 μM, but not protective at any of the three concentrations tested which suggests that the 4-OH group is necessary for high activity of the 3′,5′-dibromophenyl compounds. Also, removal of the 4′-OH group from the 5-chloro derivative (Compound 7) gives Compound 8, which is substantially less effective. Also conversion of the 4-OH to acetyl ester (Compound 9) and the 3′,4′,5′-trimethoxy derivative (Compound 3) have reduced activity as compared to the moderately active 5-chloro analog (Compound 7) and the 5-bromo (Compound 18) and 5-fluoro (Compound 32) are not effective. The results in Table 1 show also that the 3-phenylallylidenein-dolin-2-one (Compound 4) was moderately protective and non-toxic and with the exception of the 2′-nitro derivative (Compound 6) which was moderately active at 25 μM, while the 2′,6′-dichloro derivative (Compound 5) was ineffective and toxic.

The activity of CH1/4CH—C6H4 (Compound 4) and 5-nitrophenyl derivative (Compound 25) are moderately efficacious at all three concentrations used; however, the 5-F (Compound 33) and 5-Cl (Compound 12) derivatives are not effective. Substituents on the 4-position of the phenyl ring gave mixed results. For example, the 5-bromo derivative (Compound 19) was not effective. However, the 5-Cl derivative (Compound 14) was moderately active and non-toxic. Lastly, the introduction of a 4-dimethylamino group phenyl ring on the 4-dimethylamino group on the 5-bromo position (Compound 20) is not effective. However, substitution of a 2-nitro group on the benzene ring (Compound 6) results in moderate activity at high doses.

Substitution at the 5-position increases the neuroprotective activity of indolin-2-one. As shown in Table 1, the electronegative groups, i.e., 5-acetyl (Compound 34), 5-bromo (Compound 16), 5-chloro (Compound 7), and 5-nitro (Compound 21) analogs are superior to Compound 1. For example, at 1 μM and 5 μM the 5-acetyl (CH₃CO) derivative Compound 34 provides the highest survival rates (96.8% and 110.3%, respectively). However, this compound is highly toxic at 25 μM. At 5 μM, both 5-bromo (Compound 16) and 5-chloro (Compound 7) (98.7% and 91.9% survival, respectively) are highly effective and are not toxic. Finally, the 5-fluoro derivative (Compound 30) is not effective and toxic. Without wishing to bound to any theory, it is possible that electronegative substituents that are strong —I groups (electron-withdrawing by induction) but weak +R groups (electron-releasing by delocalization), such as Cl, Br, and NO₂, decrease the electron-density of the core indoline-2-one thus increasing the acidity of the 4′-hydroxy group. Further, the 5-fluoro group being not only a strong —I but a strong +R increases the electron density of the core indoline-2-one, resulting in a less acidic 4′-hydroxy group thus rendering the 5-F derivative relatively less neuroprotective.

3-(1H-Pyrrol-2-yl) (Compound 35) has no protective effect and is highly toxic at 25 μM. The efficacy of the 5-Br (Compound 37), 5-Cl (Compound 36), 5-NO₂ (Compound 39), 5-acetyl (Compound 38) and the 5-F (Compound 40) derivatives of 3-(1H-Pyrrol-2-yl) (Compound 35) are shown in Table 2. The pyrrole and thiophene derivatives are non-toxic and, more importantly, that the 5-bromo analog (Compound 37) is one of the most potent effective neuroprotective agents discovered. The remaining pyrrole derivatives have significant neuroprotective properties. Furan (Compound 45) is highly effective at 25 μM and the fluoro derivative (Compound 40) is the most effective one at 25 μM. Protective activity is provided by electron-withdrawing groups on the 5 position with 5-Br (Compound 37) and 5-NO₂ (Compound 39) having activity over 90% and, interestingly F Compound (Compound 40) has highest neuroprotective activity among these compounds. 5-Cl (Compound 36) and COME (Compound 38) have moderate protective activity. Other 5-membered heterocycles on the 3-position have interesting effects. The thiophene derivative (Compound 41) is moderately active at high doses whereas the 5-fluoro-3-(thiophen-2-yl) (Compound 44) offers low protection. Interestingly the 3-(furan-2-yl) derivative (Compound 45) is highly protective at high dosages. The configuration of the 5-membered heterocyclics does not appear to be important since both compound 35, which exist as the Z-isomer, and Compound 45, which exists as the E-isomer, have excellent activity.

One embodiment of the present invention includes a composition having the structure listed below:

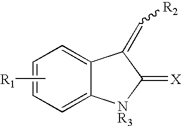

where R1 is a H, NO₂, OMe, OH, NH₂, NHMe, NMe₂, CN, CO₂H, CO₂Me, CO₂Et, CH₃, Et, N₃, F, Cl, Br, or I. X is an O, S, Se, SO, SO₂, SeO, or SeO₂. R3 is a H, Me, t-Boc, or O. R2 is a thien-2- or 3-yl, furan-2- or 3-yl, pyrrol-2- or 3-yl, pyridin-2-yl, or pyridin-3-yl. For example, R2 may be selected from:

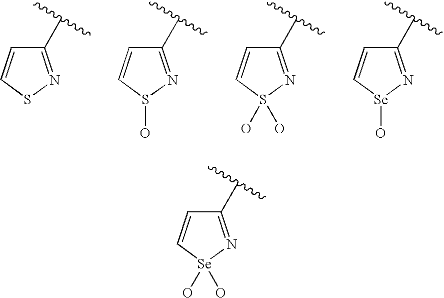

One embodiment of the present invention includes a composition having the structure listed below:

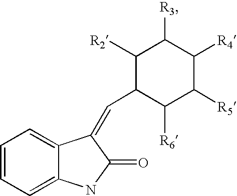

where R2′, R3′, R4′, R5′ and R6′ are independently selected from a H, NO₂, OMe, OH, NH₂, NHMe, NMe₂, CN, CO₂H, CO₂Me, CO₂Et, CH₃, Et, N₃, F, Cl, Br, and I. For example:

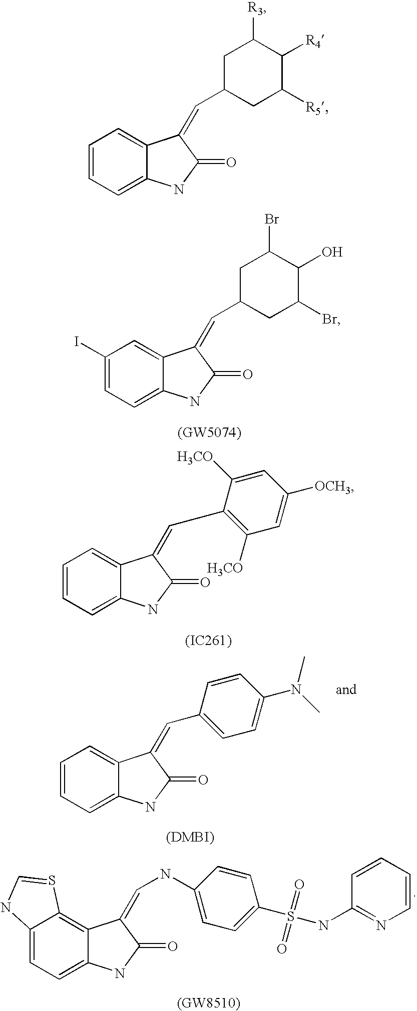

One embodiment of the present invention includes a composition having the structure listed below:

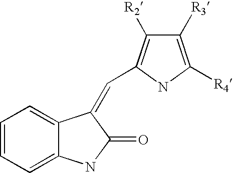

where R2′, R3′, and R4′ are independently selected from a H, NO₂, OMe, OH, NH₂, NHMe, NMe₂, CN, CO₂H, CO₂Me, CO₂Et, CH₃, Et, N₃, F, Cl, Br, I and a ring structure. For example:

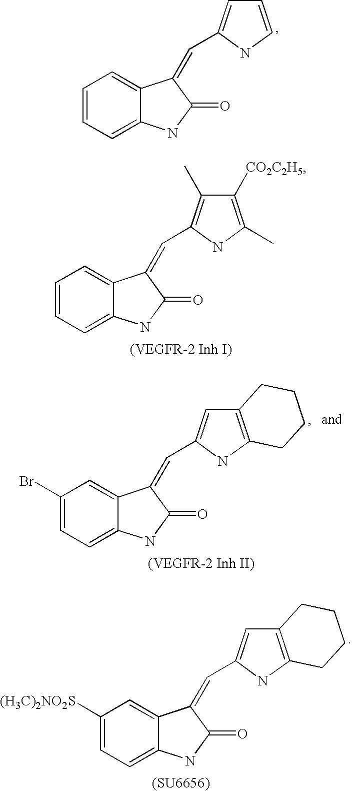

The instant invention also contemplates the optional substitution of any of the structures listed herein with a halogen, a hydrogen, an alkyl group, an alkenyl group, an aryl group, an alkoxy group, a Heterocyclyl group, a Acyl group, or an aroyl group. As such,

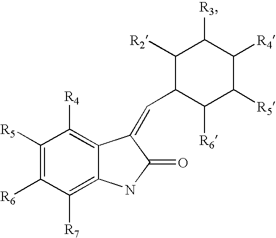

wherein R4, R5, R6, R7, R2′, R3′, R4′, R5′ and R6′ are independently selected from double bond, a halogen, a hydrogen, an alkyl group, an alkenyl group, an aryl group, an alkoxy group, a Heterocyclyl group, a Acyl group, or an aroyl group, e.g., a H, NO₂, OMe, OH, NH₂, NHMe, NMe₂, CN, CO₂H, CO₂Me, CO₂Et, CH₃, Et, N₃, F, Cl, Br, and I.

For Compound 37: The Table below shows the effect of ASK-2a on different kinases measured in vitro in the presence either 100 nM or 500 nM ASK-2a. Kinase activity is expressed as a percentage of that in control assays (without ASK-2a). The values are mean of assays performed in duplicate. Substantial inhibition of kinase activity (>20%) is highlighted by gray shading.

% ACTIVITY

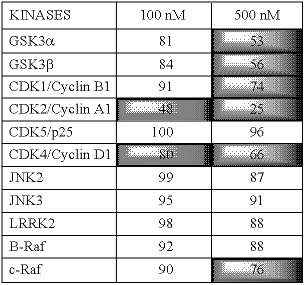

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for inhibiting the neurodegeneration process comprising the step of:
identify a patient suspected of a neurodegenerative disorder; and
providing a therapeutically effective amount of a composition sufficient to treat the neurodegenerative disorder comprising: 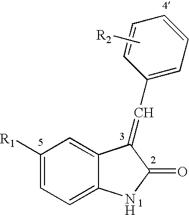
wherein R¹ is selected from the group consisting of H, halogen, nitro, and alkoxy; and R² is selected from the group consisting of 3′,5′-Br-4′-OH, 3′,5′-Br, at least one alkoxy, 2′,6′-Cl, 2-NO₂, acetyl ester, H, 4′-CH₃, 4′-OMe 4′-NMe₂, CH═CH—C₆H₅, CH═CH—C6-H4-2′NO₂, CH═CH—C₆H₄, 4′-Me, and 4′-NMe₂.

2. The composition of claim 1, wherein R2 is 2 NO₂ substitutions.

3. The method of claim 1, wherein the composition affects cerebellar granule neurons.

4. A method for inhibiting the neurodegeneration process comprising the step of:
providing a therapeutically effective amount of a composition comprising: 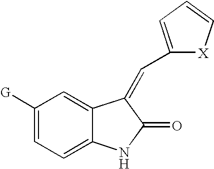
wherein G is H, Cl, Br, COMe, NO₂, F and X is NH, S or O.

5. The composition of claim 4, wherein G is Br and X is NH.

6. The composition of claim 4, wherein G is NO₂ and X is NH.

7. The composition of claim 4, wherein G is H and X is O.

8. The method of claim 4, wherein the composition affects cerebellar granule neurons.

9. A pharmaceutical composition for inhibiting the neurodegeneration process comprising:
a therapeutically effective amount a composition having the formula: 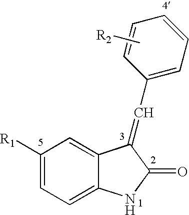
wherein R¹ is selected from the group consisting of H, halogen, nitro, and alkoxy, and R² is selected from the group consisting of 3′,5′-Br-4′-OH, 3′,5′-Br, at least one alkoxy, 2′,6′-Cl, 2-NO₂, acetyl ester, H, 4′-CH₃, 3′, 4′,5′-OMe, 4′-OMe 4′-NMe₂, CH═CH—C₆H₅, CH═CH—C6-H4-2′NO₂, CH═CH—C₆H₄, 4′-Me, and 4′-NMe₂ disposed in a pharmaceutical carrier.

10. The composition of claim 9, wherein R¹ is COCH₃.

11. The composition of claim 9, wherein R² is at least one methoxy group.

12. The composition of claim 11, wherein the at least one methoxy group is 3′,4′,5′-OMe.

13. The composition of claim 9, wherein R² is a 4′-OH.

14. The composition of claim 9, wherein the R² acetyl ester is 3′,5′-Br-4′-OAc.

15. The composition of claim 9, wherein R¹ is one of Br or Cl and R² is 3′,5′-Br-4′-OH.

16. The composition of claim 9, wherein R¹ is H and R² is 2-NO₂.

17. The composition of claim 9, wherein R¹ is NO₂.

18. The composition of claim 9, wherein R² is selected from the group consisting of 3′,5′-Br-4′-OH; 3′,5′-Br, and CH═CH—C₆H₅.

19. A pharmaceutical composition for inhibiting the neurodegeneration process comprising:
a therapeutically effective amount of a composition having the formula: 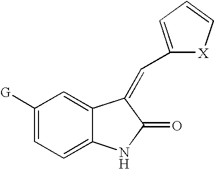
wherein G is H, Cl, Br, COMe, NO₂, F and X is NH, S or O, disposed in a pharmaceutical carrier.

20. The composition of claim 19, wherein G is Br and X is NH.

21. The composition of claim 19, wherein G is NO₂ and X is NH.

22. The composition of claim 19, wherein G is H and X is O.

23. The composition of claim 19, wherein the composition is a furan derivative.

24. A method for inhibiting a serine/threonine-specific kinase comprising the step of:
providing a therapeutically effective amount of a composition selected from (E)-3-(Furan-2′-ylmethylene)indoline-2-one, (Z)-5-Bromo-3-(thien-2′-ylmethylene)indolin-2-one, (Z)-5-Nitro-3-(thien-2′-ylmethylene) indolin-2-one, (Z)-3-(Thien-2′-ylmethylene)indolin-2-one, Z)-5-Nitro-3-(1H-pyrrol-2-yl)methylene)indolin-2-one, (Z)-5-Bromo (3-(1H-pyrrol-2′-yl)methylene)indolin-2-one, (Z)-5-Chloro-(1H-pyrrol-2′-yl)methylene)indoline-2-one, (Z)-3-(1H-Pyrrol-2′-yl)methylene)indolin-2-one, (Z)-5-Acetyl-3-(3′,5′-dibromo-4′-hydroxybenzylidene)indolin-2-one, (E)-3-Benzylidene-5-fluoroindolin-2-one, (Z)-5-Fluoro-3-(3′, 4′, 5′-trimethoxybenzylidene)indolin-2-one, (Z)-3-(3′,5′-Dibromo-4-hydroxybenzylidene)-5-nitroindolin-2-one, (E)-5-Bromo-3-(4′-(dimethylamino)benzylidene) indolin-2-one, (E)-5-Bromo-3-(4′-methoxybenzylidene)indolin-2-one, (Z)-5-Bromo-3-(3′,5′-dibromobenzylidene)indolin-2-one, (E)-5-Bromo-3-(3′,5′-dibromo-4-hydroxybenzylidene)indolin-2-one, (E)-5-Chloro-3-(4′-(dimethylamino)benzylidene)indolin-2-one, (E)-5-Chloro-3-(4′-methoxybenzylidene)indolin-2-one, (E)-5-Chloro-3-(4′-methybenzylidene)indolin-2-one, (E)-3-Benzylidene-5-chloroindolin-2-one, (E)-5-Chloro-3-(2′,6′-dichlorobenzylidene)indolin-2-one, (Z)-5-Chloro-3-(3′, 4′, 5′-trimethoxybenzylidene)indolin-2-one, (Z)-3-(3′,5′-Dibromobenzylidene)-5-chloroindolin-2-one, (E)-3-(3′,5′-Dibromo-4-hydroxybenzylidene)-5-chlorondolin-2-one, (Z-3-(2′-Nitrobenzylidene)indolin-2-one, (E)-3-(2′,6′-Dichlorobezylidene)indolin-2-one, 3-(3′-Phenylallylidene)indolin-2-one, (E)-3-(3′,5′-Dibromo-4′-hydroxybenzylidene)indolin-2-one, GW5074, IC261, DMBI, GW8510, VEGFR-2 Inh I, VEGFR-2 Inh II, and SU6656.

25. A method for the microwave synthesis of indolinones comprising the step of:
mixing an optionally substituted aldehyde with an optionally substituted indolin-2-one and piperidene;
exposing the mixture to microwaves for 1-60 minutes; and
collecting a precipitated composition.
